(19) United States
(12) Patent Application Publication
(10) Pub. No.: US 2016/0060602 A1
(43) Pub. Date: Mar. 3, 2016

(54) FUSION MIXTURE

(71) Applicant: FORSCHUNGSZENTRUM JÜLICH GMBH, Jülich (DE)

(72) Inventors: Bernd HOFFMANN, Juelich (DE); Agnes CSISZAR, Juelich (DE); Nils HERSCH, Erkelenz (DE); Rudolf MERKEL, Juelich (DE)

(21) Appl. No.: 14/778,809

(22) PCT Filed: Mar. 21, 2014
(86) PCT No.: PCT/DE2014/100098

§ 371 Date: Sep. 21, 2015

(30) Foreign Application Priority Data
Mar. 26, 2013 (DE) 10 2013 005 156.8
Jun. 11, 2013 (DE) 10 2013 009 744.4

(51) Int. Cl. C12N 5/16

(52) U.S. Cl. CPC C12N 5/16

(57) ABSTRACT

The invention relates to a fusion mixture for the lipid-containing membrane modification of an arbitrary target membrane, a cell membrane, a constituent of a cell membrane or a cell membrane separated from remaining cell constituents, in vivo or in vitro, comprising a positively charged amphipathic molecule A and an aromatic molecule B, wherein the molecule of type A and the molecule of type B are present at a ratio A:B of 1:0:02 to 1:2 mol/mol.

The invention relates to a fusion mixture for lipid-containing membrane modification of an arbitrary lipid membrane, a cell membrane, a constituent of a cell membrane or a cell membrane separated from remaining cell constituents, in vivo or in vitro, and to a method for lipid-containing membrane modification by way of fusion with this mixture.

### PRIOR ART

There is a need for methods that introduce pharmaceutically active substances into target cells for therapeutic uses.

It is known from Khalil et al. (I. A. Khalil, K. Kogure, H. Akita, H. Harashima, 2006. Uptake Pathways and Subsequent Intracellular Trafficking in Nonviral Gene Delivery. PHARMACOLOGICAL REVIEWS, Vol. 58, No. 1, 32-45) to distinguish between endocytotic and non-endocytotic uptake mechanisms for DNA. Subsequent to this review article, it has been assumed in the scientific community that the primary pathway for the uptake of DNA by way of lipoplexes (also referred to as cationic lipid-DNA complexes) takes place by endocytosis. Endocytosis describes the vesicular uptake of extracellular macromolecules. The direct uptake of macromolecules after fusion of the lipids of the lipoplexes with the cell membrane, as claimed until the mid-1990s, is consequently not tenable, and at most plays a very minor role.

The study by Friend et al. proves this assumption, in which DNA was shown in vesicles following endocytosis via electron microscopy images (Friend D S, Papahadjopoulos D, and Delos R J (1996) Endocytosis and intracellular processing accompanying transfection mediated by cationic liposomes. Biochim Biophys Acta 1278:41-50). This uptake pathway was confirmed by Weijun and Szoka Jr, cweijun Li and Francis C. Szoka Jr, 2007. Lipid-based Nanoparticles for Nucleic Acid Delivery, Pharmaceutical Research, 24, 438-449).

Cationic lipids can be used to produce cationic liposomes. It is known that the formation of cationic liposomes from starting constituents is difficult to control, as different structures can be formed from the starting substances. Thus, so as to form liposomes, cationic lipids are generally mixed in advance with neutral lipids, known as helper lipids, since cationic lipids alone do not appear to be able to ensure the formation of liposomes. The helpers used are DOPE or cholesterol, for example, as is known from the above-mentioned literature, Khalil et al. (Khalil et al., page 40, right column, first paragraph).

The helper lipid has a hydrophilic region and a hydrophobic region (in particular C₁₀-C₃₀), for example, with or without double bonds. The two regions are neutral. As a result, the high charge density and the repelling forces between positively charged molecules of type A are neutralized. This effect stabilizes the system. The use of helper lipids is therefore necessary, and also enhances the transfection efficiency of the system. The helper lipid content is set to a maximum of 70% wt/wt. Suitable molecules, include, for example, phosphatidylethanolamines, phosphatidylcholines(1,2-dioleoyl-sn-glycero-3-phosphoethanolamines, 1,2-dipalmitoyl-sn-glycero-3-phosphoethanolamines, 1,2-dimiristoyl-sn-glycero-3-phosphoethanolamines, 1,2-dielaidoyl-sn-glycero-3-phosphoethanolamines, 1,2-diphytanoyl-sn-glycero-3-phosphoethanclamines, 1,2-dilinoleoyl-sn-glycero-3-phosphoethanolamines or 1,2-dioleoyl-sn-glycero-3-phosphatidylcholines).

Positively charged fusion mixtures comprising neutral helper lipids are known from WO 2011/003406 A2, for example. The disclosed fusion mixtures are each based on a specific composition of molecules of type A (cationic lipid), molecules of type B (fluorescence marker), and molecules of type C, the neutral helper lipid. The liposomes fuse with the cell membrane on adherent cells and the plasma membrane thereof, wherein a ratio of 1:0,1:1 wt/wt is proposed for molecules of types A:B:C.

Fusogenic liposome systems are known from Csiszár et al. (Csiszár A. et al. Novel Fusogenic Liposomes for Fluorescent Cell Labeling and Membrane Modification, Bioconjugate Chemistry, 2010, 21, 537-543), which are composed of cationic (molecules of type A), neutral (molecules of type C) and aromatic lipids (molecules of type B), and in which the molecules of types A:B:C are present at a ratio of 1:0.1:1 wt/wt. The molecules of type A used are DOTAP, and the molecules of type B used are fluorescent lipophilic molecules such as DiO, DiR, Bodipy-, Lissamine rhodamine- and FITC-labeled lipids. The helper lipid C used is DOPE. These mixtures are used to create liposomes, as well as for fluorescent cell markings, protein insertion, cell membrane functionalization, active agent uptake, DNA uptake, and the like. The authors expressly point out that only the synergistic interaction of these three components results in effective fusogenic mixtures.

Kleusch at al. (Kleusch Ch. et al. Fluorescent lipids: functional parts of fusogenic liposomes and tools for cell membrane labeling and visualization, Molecules, 2011, 16, 221-250) likewise disclose cationic liposome systems composed of molecules of types A:B:C at the ratio of 1/0.1/1 wt/wt, wherein two fluorescent molecules (molecules of type B) have two consecutive functions. A non-biologically-relevant fluorescent component initially promotes rapid membrane fusion between the cellular plasma membranes and the lipid bilayers of the fusogenic liposomes, which comprise the neutral (molecules of type C) and the positively charged (molecules of type A) lipids. Following insertion into the cellular membranes, fluorescence imaging of the cell membrane and/or of the transport processes taking place there may be carried out by determining the amount of a second fluorescent component. These fusogenic liposome systems thus comprise four components (molecules of type A, molecules of type C, and two molecules of type B) and, similarly to Csiszár et al. or WO 2011/003406 with respect to three-component systems, are only disclosed in a precisely defined and fixed ratio to each other.

The disadvantages of the fusion mixtures known from the prior art are thus the narrow concentration ranges for the molecules of types A, B and C, and the synergistic interaction of at least three components to achieve an effective fusogenic mixture.

### SUMMARY OF THE INVENTION

It is the object of the invention to provide a fusion mixture, having a simple composition, for the lipid-containing membrane modification of an arbitrary lipid membrane, a cell membrane, a constituent of a cell membrane or a cell membrane separated from remaining cell constituents, in vivo or in vitro, using few types of molecules. The fusion mixture is to fuse with arbitrary lipid membranes. In particular, it should be possible for the constituents of the fusion mixture to be present across a relatively large concentration range with respect to each other so as to be able to carry out the fusion quickly and efficiently, depending on the type of cell membrane and the constituents of the fusion mixture which are used.

The fusion mixture should moreover be easy to produce.

It is a further object of the invention to provide a method for efficient membrane fusion with the fusion mixture.

### Solution to the Problem

These objects are achieved by a fusion mixture according to claim 1 and by the method according to claim 6. Advantageous embodiments in this regard will be apparent from the respective claims dependent thereon.

### DESCRIPTION OF THE INVENTION

The fusion mixture for the lipid-containing membrane modification of an arbitrary lipid membrane, such as a cell membrane, a constituent of a cell membrane or a cell membrane separated from remaining cell constituents, in vivo or in vitro, comprises a positively charged amphipathic molecule A and a molecule B, which is an aromatic molecule. The molecules of types A and B are present at a ratio of 1:0.02 to 1:2 mol/mol for this purpose.

Contrary to the prior art and existing doctrine, it was surprisingly found that molecules of type C in the form of a neutral helper lipid are not needed to reproducibly and efficiently achieve fusion of the fusion mixture with arbitrary lipid membranes.

The molecule of type A and the molecule of type B are present in the fusion mixture according to the invention at a ratio A:B of 1:0.02 to 1:2 mol/mol. Preferably, the ratio A:B is 1:0.05 to 1:1 mol/mol. This molar mixing ratio is surprisingly broad compared to known mixing ratios.

The fusion mixture according to the invention can advantageously be present in an aqueous suspension.

It was found as part of the invention and demonstrated based on experimental evidence that it is possible to reproducibly carry out membrane fusions at the specified ratio range A:B in the absence of further lipid components.

Surprisingly, it was found that an amphipathic molecule A and an aromatic compound B at the indicated ratio is a sufficient prerequisite for fusion.

The advantage of the fusion mixture according to the invention is that the mixture is easier to produce than the mixtures according to the prior art since these comprise at least three types of molecules.

By selecting the two types of molecules A and B in the indicated ranges, it is particularly advantageously achieved that membrane fusions can be reproducibly carried out, and more particularly regardless of which substance is to be moved close to or into the cell by way of the fusion mixture. The fusion takes place quickly, which is to say within a few minutes after contact of the fusion mixture with an arbitrary lipid membrane, or a constituent or a fragment thereof. The fusion can be carried out in vivo or in vitro.

The fusion mixture according to the invention can be supplemented with at least one additive Z, such as:
1. with synthetic lipid molecules and lipid mixtures;
2. with natural lipid molecules or lipid mixtures;
3. with cytoplasmic proteins and transmembrane proteins;
4. with protein/lipid mixtures;
5. with various nucleic acids (such as DNA, siRNA);
6. with a wide variety of nanoparticles (magnetic, fluorescent, and the like); and
7. with pharmacological active ingredients or other chemical or biological substances.

It should be noted that the content of the additional component Z should generally not exceed 46 mol % in the fusion mixture. Z may, of course, take on any possible intermediate value between 1 and 46 mol %, depending on the type of additive Z and the underlying fusion mixture comprising A and B. Depending on the application purpose, it is also possible for several different additives to be present in the fusion mixture according to the invention.

If multiple additives Z1 to Zn are added to the fusion mixture, these Z1 to Zn generally also have a total content of no more than 46 mol %.

The fusion mixture according to the invention can be used both for fusion with synthetic model membranes (vesicles) and for fusion with cellular membranes, including plasma membranes or cell organelle membranes, and the constituents or fragments thereof.

The membrane particles thus fused can advantageously furthermore have fusogenic properties, so that these may be used for further fusion with other membranes. Such fusions can be repeated as long as the fusogenic property of the particular (intermediate) product exists.

Fusion can be carried out in suspension and on the surface of adhered membranes.

The positively charged fusion mixture according to the invention comprises the types of molecules A and B at a ratio A:B of 1:0.02 to 1:2 mol/mol. The molecule of type B may take on any intermediate value with respect to the molecule of type A, which is to say be present at a ratio A:B of 1:0.02, 1:0.03, 1:0.04 to 1:1.96, 1:1.97, 1:1.98, 1:1.99, 1:2 and in all further intermediate values of the indicated intermediate values, such as 1:0.0025, 1:0.0032, 1:00039, 1:1.966 or 1:1.999.

Molecules of Type A

The criteria for the molecule of type A are that:

(a) the molecule includes a hydrophilic region having at least one or more positive charge, so that the overall charge of the hydrophilic part of the molecule is positive; and

(b) the molecule further includes a hydrophobic region, preferably a C10-C30 component, with or without double bonds.

The role of this molecule of type A is to bring the fusion mixture into the vicinity of the negatively charged (cell) membrane by way of electrostatic forces. Double bonds can have the advantageous effect that the membrane of the resulting liposome becomes elastic, whereby the fusion of the liposome with the cell membrane is facilitated. Suitable molecules include, for example:

1,2-dioleoyl-3-trimethylammonium propane (DOTAP);

N-(2,3-dioleyloxypropyl)-N,N,N-trimethylammonium chloride (DOTMA);

dimethyl dioctadecyl ammonium bromide (DDAB); or

(1-[2-(oleoyloxy)ethyl]-2-oleyl-3-(2-hydroxyethyl)imidazolinium chloride (DOTIM).

DOTAP (1,2-dioleoyl-3-trimethylammonium propane (chloride salt)) is mentioned as a first example.

Molecules of Type B

The molecule of type B of the fusion mixture according to the invention must be an aromatic molecule. This may mean that the molecule of type B itself represents an aromatic compound, or at least contains an aromatic group. The molecule of type B thus must include at least one delocalized electron system. Further hydrophobic and hydrophilic regions are expressly possible, but not absolutely necessary. The influence of the same on the efficiency of the fusion must be evaluated in each case.

Suitable molecules include, for example,

fluorescent dyes or dye-labeled lipids, such as:

dioctadecyloxacarbocyanine perchlorate (DiO);

1,1-dioctadecyl-3,3,3′,3′-tetramethylindotricarbocyanine iodide (DiR);

N-(4,4-difluoro-5,7-dimethyl-4-bora-3a,4a-diaza-s-indacene-3-propionyl)-1,2-dihexadecanoyl-sn-glycero-3-phosphoethanolamine, triethylammonium salt (BODIPY® FL DHPE);

2-(4,4-difluoro-5-methyl-4-bora-3a,4a-diaza-s-indacene-3-dodecanoyl)-1-hexadecanoyl-sn-glycero-3-phosphocholine (β-BODIPY® 500/510 C12-HPC); and

Texas Red® 1,2-dihexadecanoyl-sn-glycero-3-phosphoethanolamine, triethylammonium salt (Texas Red® DHPE),

or polyphenols, such as:

resveratrol, curcumin, 5-hydroxyflavone,

or vitamins, such as:

vitamin E, vitamin A or vitamin K

or cytostatic drugs, such as:

doxorubicin, paclitaxel,

or other pharmacologically active, aromatic substances.

Moreover, all chemical compounds of the molecules of type B disclosed in DE 10 2009 032 658 are suitable molecules for the type of the fusion mixture according to the invention.

All molecules that meet the criteria for molecules of type A can be arbitrarily combined with all molecules that meet the criteria for molecules of type B. This is a particular advantage of the fusion mixture according to the invention. So as to carry out fusion quickly and effectively, components A and B are combined depending on purpose and intent. A simple experimental test according to the present invention results in the necessary and/or optimal constituents of the fusion mixture with respect to molecules of types A and B for a predefined arbitrary lipid membrane and for the intended purpose of the fusion with the fusion mixture according to the invention.

For example, if DOTAB is selected as the molecule of type A, the molecule of type B may be DiO, DiR, BODIPY FL-DHPE, Texas Red-DHPE, a polyphenol, an aromatic vitamin, or an aromatic cytostatic agent, for example, whereby combinations, such as DOTAB/DiO, DOTAB/DiR, DOTAB/BODIPY FL-DHPE, DOTAB/polyphenol are obtained. DOTAB may also be replaced with another example of the molecule of type A, such as DOTIM, DDAB or DOTMA. All these combinations of molecules of types A and B are covered by the present invention, provided the molar mixing ratios range between 1:0.02 and 1:2. The molar mixing ratios can in particular also range between 1:0.05 and 1:1.

The constituents of the mixture according to the invention comprising molecules of types A and B are taken up together with the optional additives Z, preferably simultaneously in an organic solvent, preferably in chloroform, methanol, ethanol, propanol, hexane, heptane, or a mixture thereof, at the desired molar ratio. Subsequent to homogenization in organic solvent, the organic solvent is removed.

The dried homogeneous mixture is placed into an aqueous solution, preferably having a pH value around 7, and is homogenized again. The mixtures are present in storable form in this state, for example for at least 1 to 2 months at 4° C. These steps are used to prepare the method according to the invention. In a buffer, the fusion mixture can be stored for weeks

The fusion mixture advantageously forms as liposomes, comprising the added types of molecules.

Further additives Z may subsequently be admixed, such as nucleic acids (DNA, RNA), lipids, proteins, narioparticies or pharmacological active ingredients.

Lipid-containing membranes of any kind are used as fusion partners with the fusion mixtures according to the invention.

As part of the invention, it was found that the molecules of types A and B, serving as the cationic lipid A and the aromatic molecule B, can advantageously be present in any conceivable mixture according to claim 1.

According to the invention, it is advantageously consistently achieved that the partners A and B form a fusion mixture and fuse with the target membrane with high efficiency. No neutral helper lipid is necessary for this purpose.

Starting at a molar ratio A:B of 1:0.02 mol/mol in the fusion mixture according to the invention, controlled substance-transport of fusion liposomes to the cell membranes takes place. This means that the minimum supply of the molecule of type B should be present in the fusion mixture composed of the molecule of type A and the molecule of type B.

Advantageously, a molecule of type B is selected at a ratio to A at which a linear relationship can be established between the aromatic compound concentration (denoted by C) and the intensity (denoted by I) of the aromatic compound in the treated cells, for example using flow cytometry. This takes place starting at a ratio of molecules of types A:B of approximately 1:0,02 to a ratio of A:B of 1:2 mol/mol. This dependence corresponds to a general derivative Y=m*x+b, which, in the present case, is I=m*C+b where m>0.

It was found as part of the invention that a fusion of the fusion mixture with a membrane always takes place as long as a person skilled in the art selects a ratio for the two types of molecules A and B at which there is, or there is established, a linear relationship, as a function of the concentration C of molecules of type B, with the intensity I for molecules of type 8, where m>0.

The fusion mixture according to the invention fuses with the (target) membrane. During this process or subsequently, the additives Z bound potentially in the lumen or elsewhere to the fusion mixtures are released to or into the cell. This may apply similarly to molecules of type B, which, after fusion with the membrane, can be released at least partially into the interior of the cell.

This is a different method than endocytosis known from the prior art, which according to the prior art is used to transport DNA or other pharmacological active ingredients.

The target membrane may be a synthetic or a cellular membrane or membrane fraction. It may be a functionally and structurally complete membrane, which fuses with the fusion mixture during the method. Advantageously and surprisingly, using the molecules of types A and B at the indicated ratio results in the ability of all membrane types to fuse with the fusion mixture according to the invention.

According to the invention, the fusion method thus provides for the fusion mixture or the fusion liposomes to be brought in contact with a lipid-containing membrane, for example at a ratio of 1:50 to 1:200 (mixture:cell medium; v/v). After dilution, the mixture is again homogenized and placed on the cells. The contact does not have to be for a long time. Contact between the membrane and fusion mixture for several minutes, for example 1 to 10 minutes, is fully sufficient. This is a clear advantage over known methods that have considerably more slowly progressing molecule uptake by way of endocytosis.

The fusion of the fusion mixture according to the invention with the membrane particularly advantageously allows various further method steps to be combined.

By selecting a fluorescent molecule B, this can be detected in the membrane or in a cell, for example by way of fluorescence microscopy or flow cytometry. Aromatic compounds having conjugated double bonds fluoresce to varying degrees, depending on the degree of the conjugation. Highly fluorescing molecules of type B, such as the above-described fluorescent substances, are readily detectable even in the membrane by way of fluorescence microscopy.

By selecting a nucleic acid as component Z, transfection is carried out simultaneously with fusion. Fusion-bound transfections have the advantage of being considerably more efficient than transfections according to the prior art, such as with Lipofectamine.

By selecting a biotin-containing substance as component Z in the fusion mixture, particularly advantageously, biotinylation of a target membrane is carried out at the same time. This method step particularly advantageously causes target membranes to be biotinylated and prepared for further method steps. Advantageously, magnetic particles may furthermore be attached thereto, for example.

In a particularly advantageous method, one membrane type is biotinylated in a cell mixture, and another membrane type is not biotinylated in the mixture by this fusion mixture. The method may then comprise a further step in which the biotinylated membrane is bound with magnetic particles, and subsequently is separated from non-biotinylated membrane by way of magnetic force. This fusion-mediated biotinylation most particularly advantageously allows processes for purification of the mixed culture to be carried out, so as to separate the biotinylated, magnetized membranes from the non-biotinylated, non-magnetized membranes. Such purification processes particularly advantageously result in membranes that have been purified to a high degree.

In a further particularly advantageous embodiment of the invention, the (intermediate) product of the fusion mixture and target membrane is fusogenic after the fusion has been carried out. This particularly advantageously allows a further fusion to be carried out with a further lipid-containing membrane.

This advantageously allows the resulting cells and/or membranes to be fused once again with lipid membranes. Fusion steps may accordingly be repeated as long as the corresponding (intermediate) product has fusogenic properties or remains fusogenic.

These different method steps can be combined with each other by way of the selection of molecules of types A and B, and optionally Z, so that multiple methods are carried out simultaneously. Labeling of the membrane and of the DNA by way of active ingredient delivery, and the purification processes and transfections shall be mentioned by way of example.

Cell culture media with or without additives (serum) and any aqueous buffers may be used as diluents for the fusion mixture.

The fusion mixture according to the invention comprising molecules of types A and B, and optionally Z, are advantageously present in the solvent as fusion liposome.

If the molecule of type B is a fluorescent aromatic molecule, (model) membrane staining may be combined with the method according to the invention by using molecules of types A and B alone.

Depending on the type of the additionally added component(s) Z, the method according to the invention may thus be used to simultaneously:
carry out transfection, if Z is a nucleic acid (DNA, RNA).
carry out biotinylation, if Z is biotin-comprising molecules. These can be used for cell separation processes, in which different membrane types are present in biotinylated and non-biotinylated form.
introduce a pharmacologically active substance into the cell, if Z or B is such a pharmacologically active substance. In this case, the molecule of type Z may be identical to the molecule of type B. The molecule of type B is initially fused with the target membrane and then taken up into the cell.

### EXEMPLARY EMBODIMENTS

The invention will be described in more detail hereafter based on exemplary embodiments and the accompanying figures, without thereby limiting the invention.

In the drawings:

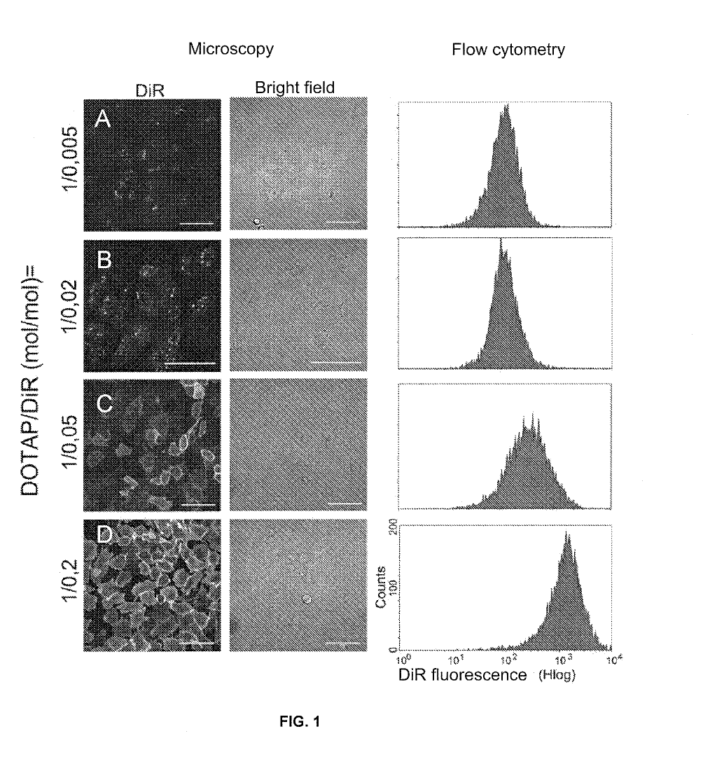

FIG. 1: CHO cells 5 minutes after treatment with aromatic compound-containing liposomes. The uptake of the liposomes was observed by way of fluorescence microscopy and bright field microscopy and quantified by way of flow cytometry; scale=50 μm. Shown are fluorescence microscopy (left column) and bright field images (center column), as well as flow cytometry measurements (right column) as a function of the ratio of molecules of types A:B used.

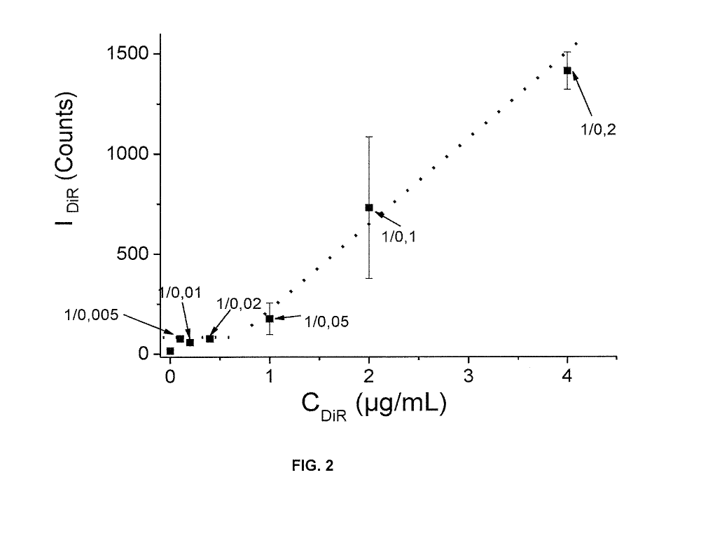

FIG. 2: DiR intensity in CHO cells 5 minutes after treatment with aromatic compound-containing liposomes. Starting at a ratio of molecules of types A:B (here: DOTAP:DiR) of 1:0.02 mol/mol, a linear relationship can be established between intensity I and concentration C, in which the mixture is preferably used, whereby liposomes are taken up by way of fusion (FIG. 1B). This process allows controlled and effective substance delivery to or into the CHO cells.

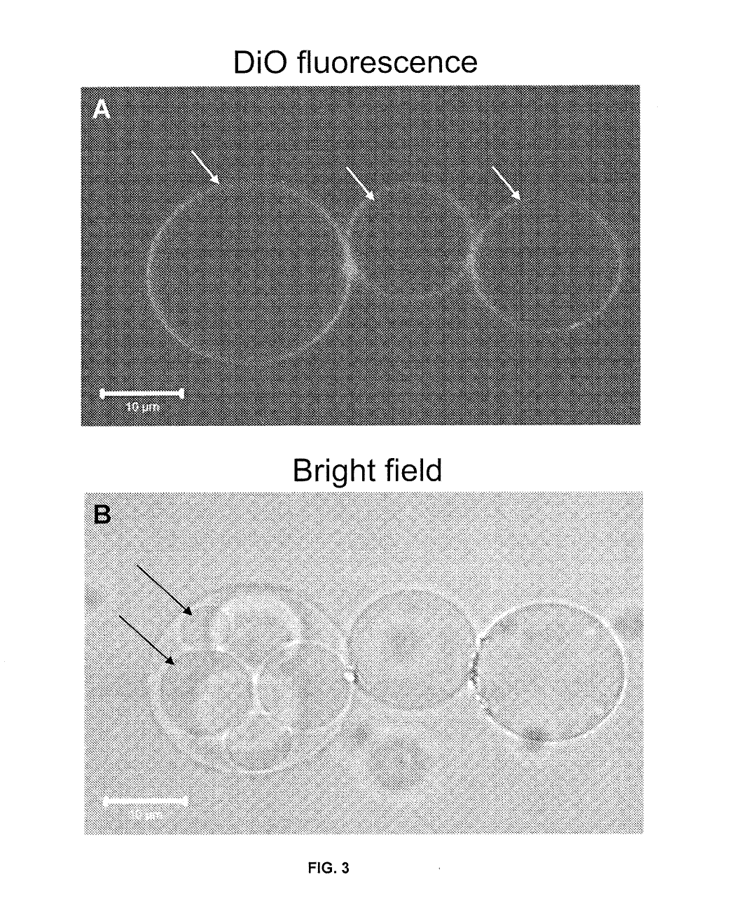

FIG. 3: DOPC giant vesicles following fusion with a fusion mixture (fluorescence image (top) and bright field image (bottom)).

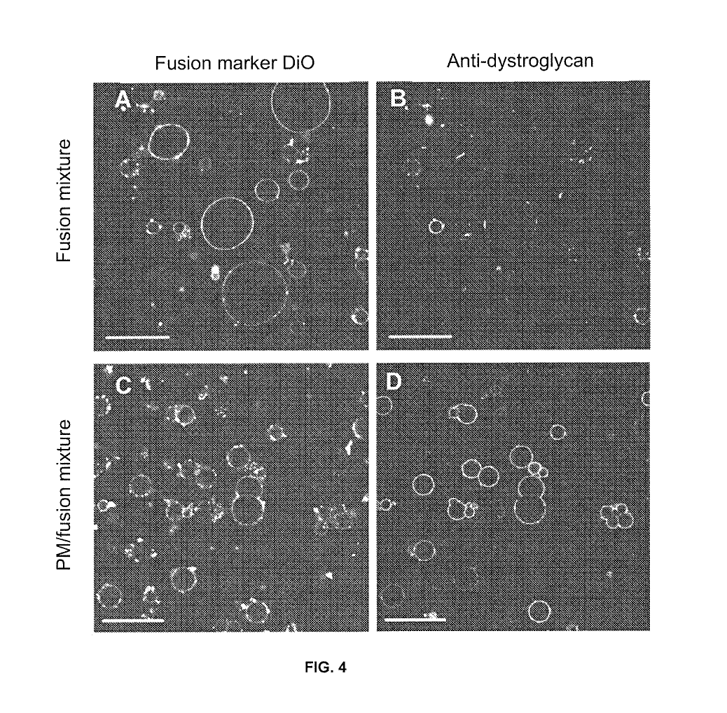

FIG. 4: DOPC giant vesicles following fusion with a fusion mixture, which contain myocyte membrane sections.

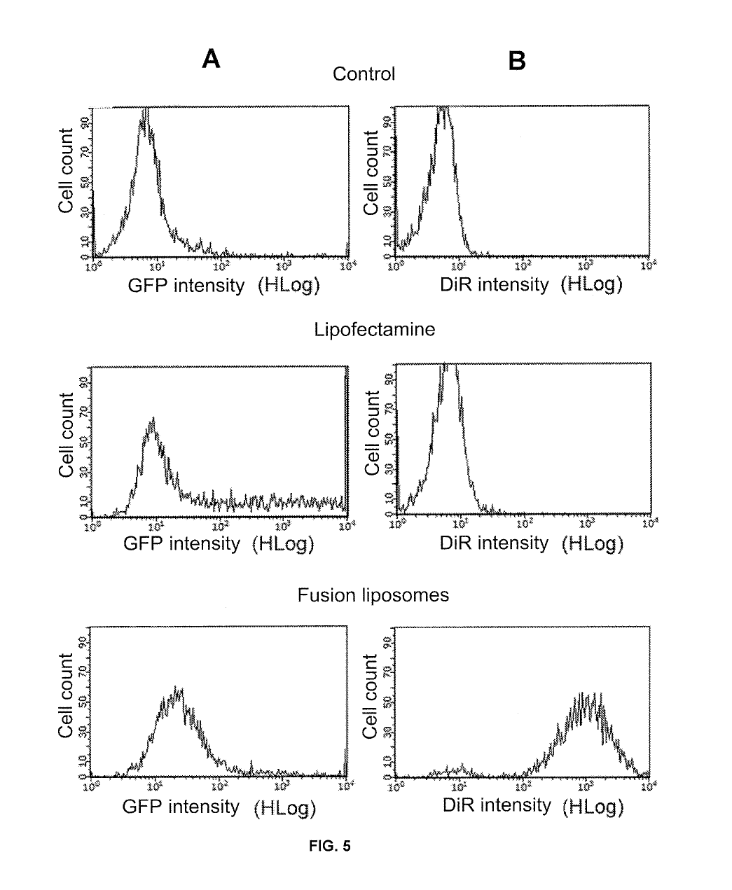

FIG. 5: GFP signal distribution (A) and DiR signal distribution (B) of transfected cells, 24 hours after treatment.

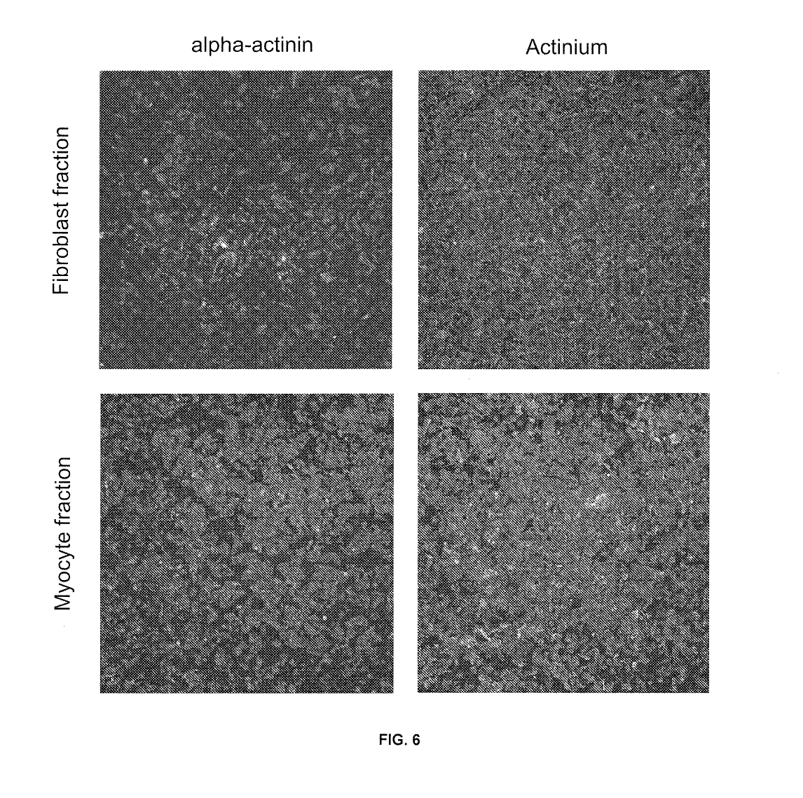

FIG. 6: Purification of primary myocytes from fibroblast/myocyte mixed culture through biotinylation of the plasma membrane of fibroblasts.

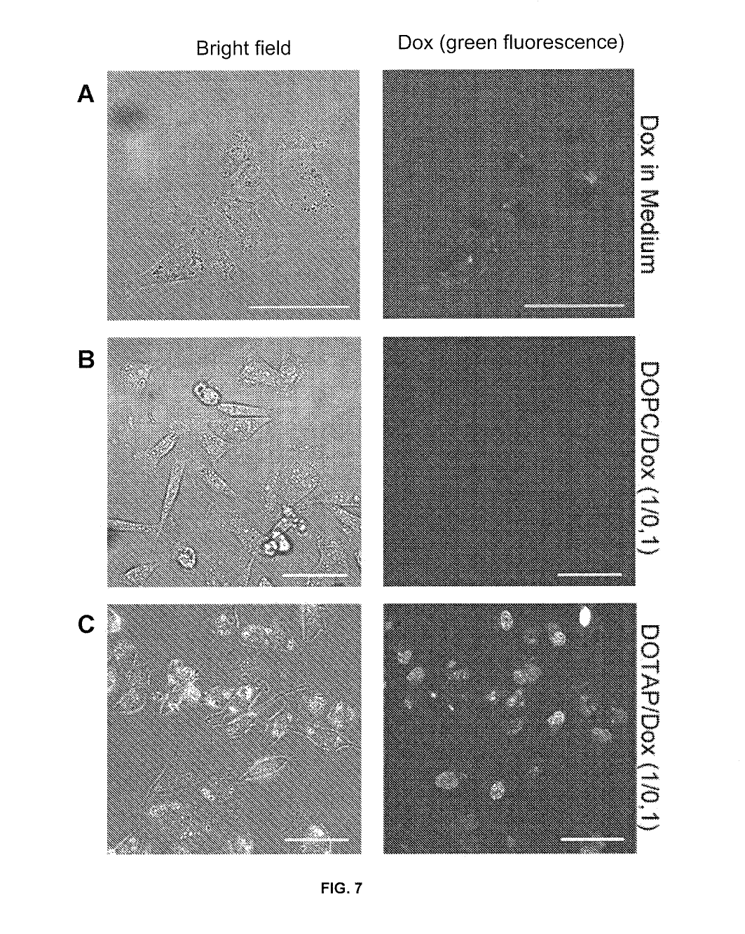

FIG. 7: MDA-MB-231 cancer cells after 10-minute treatment with free doxorubicin in the medium, with liposomal doxorubicin (DOPC:Dox of 1:0.1 mol/mol) and with doxorubicin inserted in fusion liposomes (DOTAP:Dox of 1:0.1 mol/mol). Due to the aromatic molecular structure, it is possible to detect the introduction of doxorubicin and accumulation of doxorubicin in the cell by way of fluorescence microscopy. Measuring bar=50 μm.

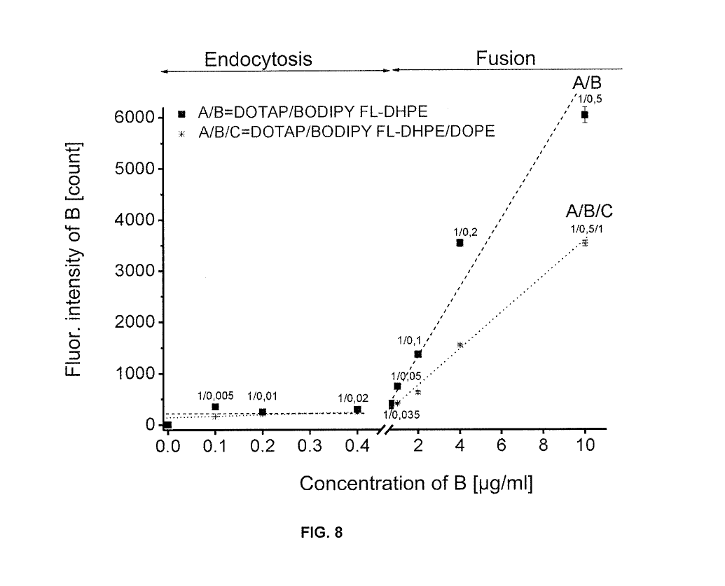

FIG. 8: BODIPY FL intensity in CHO cells 5 minutes after the treatment: comparison of the fluorescence intensity after treatment with liposomes having the composition A (DOTAP) and B (BODIPY FL-DMPP) or A (DOTAP), B (BODIPY FL-DHPE) and C (DOPE). The mixing ratios of A:B and of A:B:C were varied over a wide range.

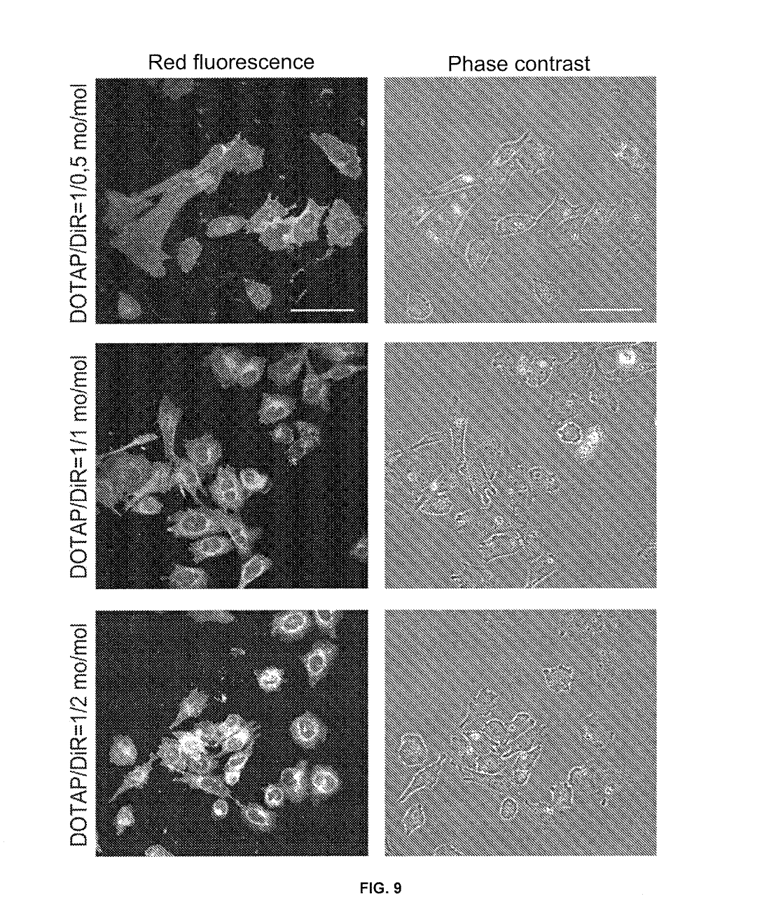

FIG. 9: CHO cells were fused with liposomes having the composition A (DOTAP) and B (DiR), Three different molar mixing ratios (A:B) were tested: 1:1; 1:1.5 and 1:2 mol/mol. Phase contrast and fluorescence images. Measuring bar 50 μm (applies to all images).

### FIRST EXEMPLARY EMBODIMENT (FIGS: 1 AND 2)

### Fusion in Cell Suspension (Fusion of Fusion Mixtures Composed of Molecules of Types A:B (DOTAP:DiR) of 1:0.005 to 1:0.2 mol/mol with CHO Cells)

The first exemplary embodiment validates the claimed molar ratios of molecules of types A and B in the fusion mixture

In the flow cytometry measurements, the labels of the X and Y axes provided in the bottom illustration also apply to the measurement results indicated above that.

The components of the fusion mixture, which here are DOTAP (molecules of type A) and DiR (molecules of type B) were mixed at a ratio of 1:0.005 to 1:2 mol/mol from 1 mg/ml parent solutions in chloroform to determine the fusion-inducing concentration range of DiR.

The result that can be noted is that, starting at a molar ratio of molecules of types A:B of 1:2 or greater (which is to say, for example, molecules of types A:B of 1:3 mol/mol), homogenization of the two substances in the fusion mixture is difficult since the substances clump. In the context of the invention, such fusion mixtures having an excessively high content of the molecule of type B in the fusion mixture should consequently be avoided.

Following the homogenization of the lipid molecule A with the aromatic compound B, the organic solvent was removed under vacuum. The dried mixture was placed in 20 mM HEPES solution having a pH of 7.4 and homogenized in an ultrasonic bath for 20 minutes.

The final concentration of the fusion mixture in the suspension was set to 2 mg/ml. After a dilution to 1/100 v/v in DMEM medium, 500 μl of the liposome suspension was added to approximately 100,000 adherent CHO cells and incubated for 5 minutes at 37° C.

FIG. 1 shows microscopic images (fluorescence of DiR and bright field images) in the left and center columns, and corresponding flow cytometry measurements in the right column. CHO cells were treated with the fusion mixture. The result shown is that after 5 minutes after treatment started, which is to say after bringing the fusion mixture in contact with the cells.

At a ratio of molecules of types A:B (DOTAP:DiR) of 1:0.005, the signal distribution corresponds to a punctiform pattern, see FIG. 1 (microscopy and flow cytometry). This indicates an endocytotic uptake process of the molecule of type B.

Starting at a molar ratio A:B (DOTAP:DiR) of 1:0.02 mol/mol, in contrast, a controlled substance-transport of fusion liposomes to the cell membranes takes place. This means that this minimum supply of the molecule of type B should be present in the fusion mixture composed of a molecule of type A and a molecule of type B.

A linear relationship is established between the aromatic compound concentration C_{Dir} and the intensity of the aromatic compound I_{Dir} in the treated cells by way of flow cytometry, and more particularly starting at a ratio of molecules of types A:B of approximately 1:0.02 up to a ratio of AB of 1:0.2 (FIG. 2).

In fact, this linear relationship occurs up to a ratio of A:B of 1:2 mol/mol (not shown). This dependence corresponds to a general derivative Y=m*x+b, which in the present case is I_{Dir}=m*C_{Dir}+b where m>0.

It was found as part of the invention that fusion of the fusion mixture with the membrane always takes place as long as a person skilled in the art selects a ratio for the two types of molecules A and B at which there is, or there is established, a linear relationship, as a function of the concentration C of molecules of type B, with the intensity I for molecules of type B, where m>0.

Below a ratio of molecules of types A:B of 1:0.02, the liposomes are taken up by way of endocytosis (see row A of FIG. 1), and the results vary randomly around an intensity value. This allows the conclusion that the positively charged liposomes enter another membrane by way of membrane fusion when aromatic molecules B are added beyond a ratio of molecules of types A:B of 1:0.02 mol/mol.

### SECOND EXEMPLARY EMBODIMENT (FIG. 3)

### Fusion with Model Membranes(Fusion of the Fusion, Mixture Composed of Molecules of Types A:B (DOTAP:DiO) with DOPC Model Membranes and a Molar Ratio A:B of 1:0.1 mol/mol)

Unilamellar giant vesicles made of 1,2-dioleoyl-sn-glycero-3-phosphocholine were swollen in a 250 mM sugar solution (pH 7.4, set with 4 mM imidazole/HCl) with a 1.3 V, 10 Hz alternating current. 100 μm vesicle solution was transferred into a measuring chamber, which was previously coated with avidin and filled with 1.8 ml 250 mM glucose solution. In addition, 100 μm of fusion vesicles (DOTAP:DiO=1:0.1 mol/mol) was added. The preparation of the fusion vesicles was carried out as in Example 1. The sample holder was covered with a cover slip, and the temperature was raised to 37° C.

FIG. 3A shows the DOPC giant vesicles (arrows) after membrane fusion has occurred. The transfer of the fluorescence from the small, fusion vesicles that cannot be resolved microscopically to the large unilamellar DOPC model membranes is proof of complete membrane fusion. The fusion additionally takes place in a controlled manner and exclusively between the fusion vesicles and the outer membrane of the DOPC giant vesicles. The inner vesicles remain visible as in FIG. 3B (bright field) compared to FIG. 3A, but uncolored (arrow).

This experiment proves targeted uptake of the fusion mixture by way of fusion into the outer membrane of a target.

### THIRD EXEMPLARY EMBODIMENT (FIG. 4)

### Fusion with Cellular Membrane Fragments—Fusion of Molecules of Types A:B (DOTAP:DiO) at a Molar Ratio of 1:0.1 mol/mol with Isolated Plasma Membrane of HL-1 Cells, and Further Fusion of these Fusogenic Membrane Fractions with DOPC Giant Vesicles

HL-1 cells (myocyte cells) were osmotically swollen in a PBS buffer at 200 mOsm. Thereafter, the cells were comminuted in a homogenizer. Cell nuclei and the mitochondria could be separated from the remaining membrane by centrifugation. The residual fraction contains the cellular plasma membranes with dystroglycan, ER membranes and lysosomal membranes. This fraction was placed in a buffer solution of 250 mM sugar/imidazole/HCl.

This was fused with fusion liposomes composed of DOTAP/DiO (1:0.1 mol/mol) at a volume ratio of 1/10 to generate plasma membrane-containing fusion liposomes.

A second fusion (C, D) was started between the plasma membrane-containing fusion liposomes and DOPC giant vesicles.

All DOPC giant vesicles were produced without fluorescence markers.

FIG. 4A shows giant vesicles after fusion with a fusion mixture composed of DOTAP and DiO (green channel).

FIG. 4B shows the same giant vesicles after fusion with a fusion mixture composed of DOTAP and DiO and antibodies against dystroglycan (red channel).

In the case of successful fusion, the green color of DiO was transferred to the DOPC giant vesicles (green channel, FIG. 4A). The red channel (4B) showed no signal, that is, absence of dystroglycan (negative control).

The PM fraction of the myocytes was fused with a fusion mixture of DOTAP (molecules of type A) and DiO (molecules of type B) (first fusion). The plasma membrane protein dystroglycan was then transferred into the DOPC giant vesicle membrane by way of fusion (second fusion).

This was demonstrated by Immunostaining using the anti-dystroglycan ATTO 633 antibody (red channel).

FIG. 4 shows the DOPC giant vesicles after the fusion with fusion liposomes without (A, B) and with (C, D) plasma membrane component. The green color of DiO was transferred to the giant vesicles by way of membrane fusion. The plasma membrane protein dystroglycan was likewise introduced into the vesicle membrane by fusion. This was demonstrated with red anti-dystroglycan staining.

### FOURTH EXEMPLARY EMBODIMENT (FIG. 5)

### Transfection—Fusion of DOTAP/DiR/DOPE (1:0.1:1 mol/mol)/Plasmid Complex with CHO Cells in Suspension

The components of fusion liposomes, here DOPE/DOTAP/DiR, were mixed at a ratio of 1/1/0.1 mol/mol from 1 mg/ml parent solution in chloroform. Following homogenization of the lipids, the organic solvent was removed under vacuum. The dried mixture was placed in a buffer solution of 20 mM HEPES/NaOH, pH of 7.4, and homogenized in an ultrasonic bath for 20 minutes. The final concentration of the fusion mixture was set to 2 mg/ml.

20 μl fusion mixture was again homogenized in an ultrasonic bath with 2 μg EGFP plasmid for 10 minutes. The fusion mixture/plasmid complex was subsequently diluted 1:100 v/v with PBS, added to approximately 100,000 CHO cells (suspension), and incubated for 20 minutes at 37° C. Both the EGFP expression and the fusion efficiency were measured by way of flow cytometry. Untreated CHO cells and CHO cells transfected in the traditional way via Lipofectamine 2000 (Invitrogen) were used as controls.

FIG. 5A shows the GFP intensity distribution of the three samples. The two transfected samples showed a considerably shifted intensity profile compared to the untreated control. The difference is particularly apparent in the profile distribution. The transfection efficiency of the Lipofectamine sample is approximately 60% and shows drastically varying GFP intensities. This means that there are cells that have high GFP expression, while other cells express only very little GFP. Such cells are usually very difficult to analyze or not meaningful.

In contrast, the transfection efficiency of the cells transfected by the method according to the invention with fusion mixture is more than 75%. The advantage over traditional transfection is the homogeneous expression level in ail cells. FIG. 5B shows that the transfection efficiency of the cells treated with the fusion liposome/plasmid complex correlates with the fusion efficiency. The fusion efficiency was calculated based on the DiR signal distribution and has a value of 90 to 95%.

### FIFTH EXEMPLARY EMBODIMENT (FIG. 6)

### Plasma Membrane Biotinylation by Fusion of DOTAP:DiO:CapBiotin-DHPE (1:0.05:0.1 mol/mol) with Primary Cardiac Fibroblasts in Suspensions

The components of the fusion mixture composed of A:B:Z (DOTAP:DiO:capBiotin-DHPE as component Z) were mixed at a ratio of A:B:Z of 1:0.05:0.1 mol/mol from 1 mg/ml parent solutions in chloroform and homogenized. The fusion liposomes were moreover prepared as described in Example 1.

10 μl of the biotinylated fusion liposomes was diluted 1:100 in DME medium, and the cardiac fibroblasts (1 to 6 million) were subsequently resuspended in the fusion mixture. After incubation for 1 to 3 minutes, which was sufficient time for fusion of the fibroblasts while the myocyte population was not yet fused, the cells were washed two times with PBS, pelletized, and resuspended in 100 μl magnetic Anti-Biotin Beads solution and incubated for 20 minutes at 4° C. All fused fibroblasts were labeled with magnetic beads via biotin-anti-biotin binding.

This cell population was separated from the suspension with the aid of a chromatography column located in a magnetic field. The pure myocyte population was not separated by the magnetic field and was collected.

FIG. 6 shows the two cell populations. The actinium staining labels both cell types (myocytes and fibroblasts), and the myocyte-specific antibody alpha-actinin exclusively makes myocytes visible in the immunostaining. The ratio of the starting culture is approximately 50:50 and shifts to approximately 5% fibroblasts to 95% myocytes as a result of the described method.

At present, no method is available to obtain similarly pure myocyte cultures. The method of fusion-controlled biotinylation can be applied to other cell types.

### SIXTH EXEMPLARY EMBODIMENT (FIG. 7)

### Doxorubicin Delivery of Fusion Liposomes in Cancer Cells

Doxorubicin is used today as a pharmaceutical product in chemotherapy. It intercalates into the DNA in the cell nucleus and disrupts DNA synthesis. Doxorubicin has an aromatic molecular structure and was tested here directly as the molecule of type B, and as a constituent of positively charged liposomes with respect to the induction of membrane fusion between the liposomes membrane and cell membrane. The following experiments were conducted subsequent to prior fusion, so as to demonstrate the inward transfer of doxorubicin into cancer cells.

The components of fusion liposomes, DOTAL (molecules of type A) and Doxorubicin (molecules of type B), were mixed at a ratio of 1:0.1 mol/mol from 1 mg/ml parent solutions in chloroform.

Following homogenization of the lipid molecules with the aromatic doxorubicin, the organic solvent was removed under vacuum. The dried mixture was placed in 20 mM HEPES solution having a pH of 7.4 and homogenized in an ultrasonic bath for 20 minutes. The final concentration of the fusion mixture was set to 2 mg/ml. After a dilution of 1/100 v/v in DMEM medium, 500 μl of the fusion mixture was added to approximately 100,000 adherent MDA-MB-231 cancer cells and incubated for 10 minutes at 37° C.

A further cell sample was incubated with the same amount of doxorubicin (2 μg) in culture medium without fusion mixture for control purposes. The conventional dosage is 1 mg/ml.

In addition, doxorubicin was introduced into neutral DOPC liposomes (without molecules of type A) (DOPC:Dox at a ratio of 1:0.1 mol/mol) and added to adherent cells in the same concentration as in the other sample (2 μg/ml). The doxorubicin uptake was observed in all cases by way of fluorescence microscopy. The results were then compared to each other.

FIG. 7 shows the microscopic images in the bright field (left column) and the corresponding fluorescence microscopy images (green channel, right column) after 10-minute incubation. The most effective doxorubicin uptake was established with the use of fusion liposomes (FIG. 7C). Here, all cells used showed an intensive doxorubicin fluorescent signal in the cell nucleus.

Such rapid inward transfer of doxorubicin into the nucleus has not previously been known. The most frequently employed doxorubicin incubation times range between 24 and 72 h.

The control with free doxorubicin in the medium (FIG. 7A) shows a low fluorescent signal, and the DOPC/Dox mixture shows no signal (FIG. 7B). Endocytosis-mediated substance transport processes generally require a longer time for completion (hours to days).

FIG. 7C shows the intercalation of Dox with the DNA in the cell nucleus and hence the rapid uptake and use as a pharmacologically active substance, and at the same time the use as a DNA dye.

This method makes it possible to treat cancer cells with a cytostatic agent in just a few minutes.

### SEVENTH EXEMPLARY EMBODIMENT (FIG. 8)

### Comparison of the Fluorescence Intensity between Liposomes, Produced from Molecules of Types A (DOTAP) and B (BODIPY FL-DHPE), and Liposomes Produced from Molecules of Types A (DOTAP), B (BODIPY FL-DHPE) and C (DOPE), Each with Different Molar Fractions of Type B

In this exemplary embodiment, the fluorescence intensities of CHO cells after treatment with liposomes of types A/B and with liposomes of types A/B/C are directly compared to each other. When relatively low concentrations of the molecules of type B are used, which is to say in the concentration range of 0.1 to 0.4 μg/ml, or molar mixing ratios of 1/0.005 to 1/0.02 for A/B and 1/0.005/1 to 1/0.02/1 for A/B/C, the uptake of the liposomes takes place via endocytosis, and no difference is discernible between the A/B type and the A/B/C type in terms of fluorescence intensities. Furthermore, the intensities are in the range up to 1/0.02 for A/B, and up to 1/0.02/1 for A/B/C, which is approximately the same low level. As a result, almost no linearity exists between the aromatic compound concentration used (molecules of type B) and the measured intensity of the aromatic compound (FIG. 8). Starting at a molar mixing ratio of A:B (DOTAP:BODIPY FL) of 1:0.02 mol/mol and of A:B:C (DOTAP:BODIPY FL:DOPE) of 1:0.02:1 mol/mol, membrane fusion is induced in both instances. However, the fusion, and hence the transfer of molecules, takes place considerably more efficiently with liposomes that are composed only of molecules of types A and B, compared to the liposomes that are composed of 3 components (A/B/C). FIG. 8 shows this for molar mixing ratios of 1/0.035 and 1/0.035/1 to 1/0.05 and 1/0.05/1. The considerable superiority of A/B liposomes compared to A/B/C liposomes is clearly evident, even though the amount of molecules of type B used is identical in both cases, as can be understood from the concentrations of B, expressed in μg/ml, in FIG. 8. Starting at a molar mixing ratio A:B of 1:0,02 mol/mol, fusion and controlled substance-transport from fusion liposomes to the cell membranes take place, accompanied by a linear relationship between the aromatic compound concentration and the intensity of the aromatic compound in the treated cells. This is shown in FIG. 8 up to a molar mixing ratio A:B of 1:0.5, wherein the mixing ratio A:B can be expanded up to 1:2, as is shown, for example, in FIG. 9 in connection with the eighth exemplary embodiment.

Contrary to the preceding exemplary embodiments 1 to 7, in which DiO, DiR or doxorubicn was used as the molecule of type B, a further type B (BODIPY FL-DHPE ), was successfully tested in the present exemplary embodiment.

### EIGHTH EXEMPLARY EMBODIMENT (FIG. 9)

### Fusion with Adherent CHO Cells (Fusion of Fusion Mixtures Composed of Molecules of Types A:B (DOTAP:DiR) of 1:0.5 to 1:2 mol/mol)

Compared to the first exemplary embodiment, the eighth exemplary embodiment validates higher contents of the molecule of type B over the molecule of type A. The phase contrast images show that the cells maintain vitality after treatment, even with high contents of the molecule of type B, and do not undergo any morphological changes. Biocompatibility is thus very high. The fluorescence images illustrate the increasing introduction of cellular dye as the dye content in the liposomes increases.

1. A fusion mixture for the lipid-containing membrane modification of an arbitrary lipid membrane, a cell membrane, a constituent of a cell membrane or a cell membrane separated from remaining cell constituents, in vivo or in vitro, comprising a positively charged amphipathic molecule A and a molecule B, wherein the molecule of type B is an aromatic molecule, and the molecule of type A and the molecule of type B are present at a ratio A:B of 1:0.02 to 1:2 mol/mol.

2. The fusion mixture according to claim 1, wherein the fusion mixture is present in an aqueous solution.

3. The fusion mixture according to claim 1, comprising at least one additive Z.

4. The fusion mixture according to claim 3, comprising synthetic lipid molecules, natural lipid molecules, cytoplasmic proteins, transmembrane proteins, protein/lipid mixtures, nucleic acids, nanoparticles (magnetic, fluorescent, and the like) or pharmacological active ingredients, or mixtures thereof, as the additive Z.

5. The fusion mixture according to claim 4, comprising an additive Z content of no more than 46 mol %.

6. A method for lipid-containing membrane modification of an arbitrary lipid membrane, a cell membrane, a constituent of a cell membrane or a cell membrane separated from remaining cell constituents, in vivo or in vitro, comprising bringing the lipid-containing membrane in contact with the fusion mixture according to claim 1 and the fusion mixture fusing with the membrane.

7. The method according to claim 6 wherein a transfection is simultaneously carried out, by way of selecting a nucleic acid as the component Z.

8. A method according to claim 6, wherein biotinylation of a target membrane is simultaneously carried out by way of selecting a biotin-containing substance as the component Z in the fusion mixture.

9. The method according to claim 8, wherein a further membrane is not biotinylated by this fusion mixture.

10. The method according to claim 9, comprising a step in which biolinylated membrane is bound with magnetic particles and subsequently is separated from ion-biotinylated membrane by way of magnetic force.

11. A method according to claim 6, wherein the product of the fusion mixture and target membrane is fusogenic after the fusion has occurred, and a further fusion is carried out with further lipid-containing membrane.

12. A method according to claim 6, comprising selecting a pharmacologically active substance as the component Z, which is taken up into the cell after fusion into the membrane.

13. The method according to claim 12, comprising selecting a component Z, which in addition to the pharmacological action comprises an aromatic constituent.
